SEQUENCE LISTING (30 sequences)

SEQ ID NO: 1 - DNA, 9 bp, Artificial Sequence
  A core sequence of decoy oligonucleotides binding to transcroption factor CREB
stgacgtmr

SEQ ID NO: 2 - DNA, 8 bp, Artificial Sequence
  A core sequence of decoy oligonucleotides binding to transcroption factor AP-1
stgastma

SEQ ID NO: 3 - DNA, 8 bp, Artificial Sequence
  A core sequence of decoy oligonucleotides binding to JUN/ATF heterodimer
ttacctca

SEQ ID NO: 4 - DNA, 7 bp, Artificial Sequence
  A core sequence of decoy oligonucleotides binding to MYC protein
tctctta

SEQ ID NO: 5 - DNA, 12 bp, Artificial Sequence
  A core sequence of decoy oligonucleotides binding to MYC/MAX heterodimer
raccacgtggty

SEQ ID NO: 6 - DNA, 23 bp, Artificial Sequence
  a single-stranded oligonucleotides that forms a hairpin structure and binding to transcription factor CREB
gctgacgtcagactgacgtcagc

SEQ ID NO: 7 - DNA, 24 bp, Artificial Sequence
  A single-stranded oligonucleotides that binding seq.8 and forms a cruciform structure,and then binding to transcription factor CREB
gtttcggggtgacgtcaccctttc

SEQ ID NO: 8 - DNA, 24 bp, Artificial Sequence
  A single-stranded oligonucleotides that binding seq.7 and forms a cruciform structure,and then binding to transcription factor CREB
gaaagggacgtacgtccgcgaaac

SEQ ID NO: 9 - DNA, 23 bp, Artificial Sequence
  A double-stranded linear oligonucleotides binding to transcription factor CREB
agattgcctgacgtcagagagct

SEQ ID NO: 10 - DNA, 20 bp, Artificial Sequence
  A double-stranded linear oligonucleotides binding to transcription factor AP-1
cgcttgctgactcagccgga

SEQ ID NO: 11 - DNA, 21 bp, Artificial Sequence
  A single-stranded oligonucleotides that forms a hairpin structure and binding to transcription factor AP-1
tcagtactgactcagtactga

SEQ ID NO: 12 - DNA, 42 bp, Artificial Sequence
  A single-stranded oligonucleotides that forms a dumbbell structure and binding to transcription factor AP-1
actctctcagtctgactcatgctctcagcatgagtcagactg

SEQ ID NO: 13 - DNA, 22 bp, Artificial Sequence
  A single-stranded oligonucleotides that forms a hairpin structure and binding to transcription factor AP-1
tcacggtatgactcatccgtga

SEQ ID NO: 14 - DNA, 21 bp, Artificial Sequence
  A single-stranded oligonucleotides that forms a hairpin structure and binding to transcription factor AP-1
cacggtctgactcagaccgtg

SEQ ID NO: 15 - DNA, 23 bp, Artificial Sequence
  A single-stranded oligonucleotides that forms a hairpin structure and binding to transcription factor AP-1
tgagtcagtgactcactgactca

SEQ ID NO: 16 - DNA, 21 bp, Artificial Sequence
  A single-stranded oligonucleotides that forms a hairpin structure and binding to transcription factor AP-1
ctccggctgactcagccggag

SEQ ID NO: 17 - DNA, 23 bp, Artificial Sequence
  A single-stranded oligonucleotides that forms a hairpin structure and binding to JUN/ATF heterodimer
gttacctcagcccgtgaggtaac

SEQ ID NO: 18 - DNA, 21 bp, Artificial Sequence
  A single-stranded oligonucleotides that forms a hairpin structure and binding to JUN/ATF heterodimer
gttacctcagcctgaggtaac

SEQ ID NO: 19 - DNA, 22 bp, Artificial Sequence
  A single-stranded oligonucleotides that forms a hairpin structure and binding to JUN/ATF heterodimer
gttacctcacgcgtgaggtaac

SEQ ID NO: 20 - DNA, 20 bp, Artificial Sequence
  A single-stranded oligonucleotides that forms a hairpin structure and binding to JUN/ATF heterodimer
cgttacctcatgaggtaacg

SEQ ID NO: 21 - DNA, 20 bp, Artificial Sequence
  A double-stranded linear oligonucleotides binding to JUN/ATF heterodimer
attgccttacctcagagagc

SEQ ID NO: 22 - DNA, 26 bp, Artificial Sequence
  A double-stranded linear oligonucleotides binding to MYC protein
tcaccatctcttatgcggttgaatag

SEQ ID NO: 23 - DNA, 25 bp, Artificial Sequence
  A single-stranded oligonucleotides that forms a hairpin structure and binding to MYC protein
tatgactaatctcttattagtcata

SEQ ID NO: 24 - DNA, 26 bp, Artificial Sequence
  A single-stranded oligonucleotides that binding seq.25 and forms a stem-loop structure,and then binding to MYC protein
tctcttagctctcttagcctctctta

SEQ ID NO: 25 - DNA, 17 bp, Artificial Sequence
  A single-stranded oligonucleotides that binding seq.24 and forms a stem-loop structure,and then binding to MYC protein
taagagaggctaagaga

SEQ ID NO: 26 - DNA, 19 bp, Artificial Sequence
  A single-stranded oligonucleotides that binding seq.27 and forms a hammer structure,and then binding to MYC protein
gagtctcttactcccgcgg

SEQ ID NO: 27 - DNA, 17 bp, Artificial Sequence
  A single-stranded oligonucleotides that binding seq.26 and forms a hammer structure,and then binding to MYC protein
ccgcgggtctcttagac

SEQ ID NO: 28 - DNA, 24 bp, Artificial Sequence
  A single-stranded oligonucleotides that forms a hairpin structure and binding to MYC/MAX
cacggagaccacgtggtctccgtg

SEQ ID NO: 29 - DNA, 24 bp, Artificial Sequence
  A single-stranded palindromic oligonucleotides that self-anneal to form double-stranded dimer,and then binding to MYC/MAX
gaagcagaccacgtggtctgcttc

SEQ ID NO: 30 - DNA, 24 bp, Artificial Sequence
  A single-stranded palindromic oligonucleotides that self-anneal to form double-stranded dimer,and then binding to MYC/MAX
aaccacgtggttaaccacgtggtt

(19) United States
(12) Patent Application Publication
(10) Pub. No.: US 2004/0204374 A1
(43) Pub. Date: Oct. 14, 2004

(54) Cis-element decoys useful as anti-tumor therapeutics

(76) Inventors: Xuegen Wang, NanJing (CN); Qing Ye, NanJing (CN); Wuling Chen, NanJing (CN); Ying Feng, NanJing (CN)

Correspondence Address: Raymond Y. Chan, Suite 128, 108 N. Ynez Ave., Monterey Park, CA, 91754, US

(21) Appl. No.: 10/411,692

(22) Filed: Apr. 11, 2003

(51) Int. Cl. A61K 48/00; C07H 21/04

(52) U.S. Cl. 514/44; 435/375; 536/23.2

(57) ABSTRACT

An anti-cancer or oncogene switching therapeutics that employ sequence-specific nucleic acid decoys for competitive binding of transcription factors. These nucleic acid decoys can competitively bind to transcription factors regulating the expression of oncogenes including CREB, JUN/FOS, JUN/ATF and MYC/MAX in the cell, leading to the inhibition of tumor cell growth. As a result, they can be developed into anti-cancer therapeutic drugs.

### BACKGROUND OF THE PRESENT INVENTION

1. Field of Invention

The present invention relates to compositions of synthetic oligonucleotides as cis-element decoys for competitive binding of targeted transcription factors and their applications in the field of tumor biology and anti-tumor therapeutics.

2. Description of Related Arts

One of the characteristics of tumor cells is the overexpression or amplification of proto-oncogenes. For example, C-MYC is found to be overexpressed in leukemia, lymphoma and sarcoma and is amplified to different extents in leukemia, breast cancer, stomach cancer, small cell lung cancer, colon cancer, neuroblastoma, and glioblastoma. Other examples include the overexpression of C-FOS in osteosarcoma and lymphoma and C-JUN is found to be highly overexpressed in small cell lung cancer, non-small cell lung cancer, colon and rectal cancers. In addition, C-MYC and C-JUN are found to be overexpressed at tumor's initiation and incubation stages (to be linked to the appearance of tumors). Finally, the amplification of C-MYC is one of the pre-requests for tumorigenesis.

Another characteristics of tumor cells is the loss of cell cycle control and the proper reaction to the cell growth factors. The heterodimer MYC/MAX binds to E-box to activate the expression of itself and its down stream genes such as CYC25A, leading to the formation of the cell growth phenotype. Together with E2F, the heterodimer can also activate CyclinA and cyclinE, a key event for the G1 to S phase transition in the cell cycle. C-MYC is found to activate the expression of telomerase and play an important role in tumor formation, while C-JUN and C-FOS are important in the down stream nucleus phase signal transduction of the RAS pathway. Furthermore, C-MYC, C-FOS and C-JUN are important in cell apoptosis, genome stability and cell maturation. Therefore, C-MYC, C-FOS and C-JUN can act as drug targets. By fixing the abnormal expression of these three proto-oncogenes, the cancerous hypergrowth phenotype can potentially be transformed into a benign and normal phenotype.

Proto-oncogenes and tumor suppressor genes coexist in normal and tumor cells. The difference between normal and tumor cells lies in the expression levels of the two types of genes. In the tumor cells, the proto-oncogenes are overexpressed by 2-10 times while the tumor suppressor genes are inactivated. These abnormalities are closely related to the binding status, type and quantity of the cistrons of the regulatory regions of said genes. Removing the abnormalies by changing the gene regulatory states is a novel approach for oncogene therapy.

Transcription factor decoys utilizing the specific DNA sequences for binding to transcription factors in order to change the gene expression levels, therefore leading to changes in cell phenotypes, can be developed into an effective tumor therapy.

Studies in DNA-protein interactions form the experimental basis for developing nucleic acid decoys as therapeutics. After a short piece of in vitro synthesized DNA with a specific sequence binds to certain protein factor extracted from the nucleus, the resulting DNA-protein complex band is shifted compared to the free DNA in polyacrylamide gel electrophoresis. Since Bielinska (1990) proposed the application of double-stranded oligonucleotides to regulate gene expression in the 90's, nucleic acid decoys have been initially used in cardiovascular drug development. For example, nucleic acid decoy therapy targeting the E2F factor for the treatment of inner membrane growth after CABG has entered clinical trials (Mann et. al, 1999). Nucleic acid decoys targeting NF-kappa ε for the treatment of ischemia was also reported (Sawa, 1997; Tounita, 1998). The application of nucleic acid decoys in tumor therapy is in its early stages. Nucleic acid decoys for the CREB factor (Cho-chong, 1999) has been tested in multiple tumor cell lines and in animal models. Nucleic acid decoys for ER have been used to inhibit breast cancer cell growth (Piva R, et. al, 200). Nuclei acid decoys for NF-kappa have been found to inhibit mouse tumor growth both in vitro and in vivo.

### SUMMARY OF THE PRESENT INVENTION

The present invention covers oncogene switching therapeutics that are composed of sequence-specific nucleic acid decoys. These kinds of nucleic acids can competitively combine with certain transcription factors, and regulate the expression of proto-oncogenes such as C-FOS, C-JUN and C-MYC, leading to the inhibition of tumor cell growth and attaining the objective of tumor treatment.

The primary objective of the present invention is to develop pharmaceutical compositions comprising one or a combination of the nucleic acid decoys.

Another objective of the present invention is to apply said nucleic acid decoys as therapeutics for anti-cancer drugs.

These oncogene switching therapeutics use the specific sequences that bind to transcription factors as the core sequences of the nucleic acid decoys. In order to increase the drugs' stability and affinity for drug targets, the core sequences are expanded in both directions with more nucleotides so that the longer sequences can form three-dimensional structures.

The present invention discloses several pharmaceutical composition of nucleic acid decoys molecules specifically target the drug targets of transcription factors such as AP-1, CREB, MYC/MAX and JUN/ATF transcription machinery. Said decoys are shown to inhibit tumor cell growth and possess great promises as anti-cancer drug candidates. The said pharmaceutical composition can be combined with carriers consisted of saline, buffer, liposome and polymers to be made into formulations consisted of injectables, oral capsules and tablets, bolus, suppository and skin applicants. Said pharmaceutical composition can also be used in combination with chemotherapeutics and radiation therapies to enhance their efficacies.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a diagram illustrating growth inhibition of NCI-H460 tumor cells by KGCD series nucleic acid decoys. Growth inhibition of NCI-H460 tumor cells by 6 KGCD series nucleic acid decoys is shown in FIG. 1. The cell survival rate is calculated by SRB staining and measuring the OD values, with blank control as 100%. CRE represents positive control, C is the negative control and the rest 6 groups are KGCD series decoys. FIG. 1 shows NCI-H460 cell growth inhibition to various degrees by the decoys, with K101a and K102 exhibiting the most severe growth inhibition.

FIG. 2 is a diagram illustrating growth inhibition of various tumor cell lines by nucleic acid decoys K101a and K102. K101a and K102 are used to treat the following tumor cell lines: NCI-H460 (lung cancer), CNE (nasopharyngeal carcinoma), U251 (neuroglioblastoma), MCF-7 (breast cancer), BEL-7402 (liver cancer) and normal cell lines L-02 (liver cell) and NIH3T3. The column with the faintest color represents negative control (mismatch sequence), showing little influence on cell growth. K101a and K102 inhibit the growth of all the tumor cell lines to different degrees with the growth inhibition to U251 and NCI-H460 the most severe. The decoys have little effective on the growth of normal cells.

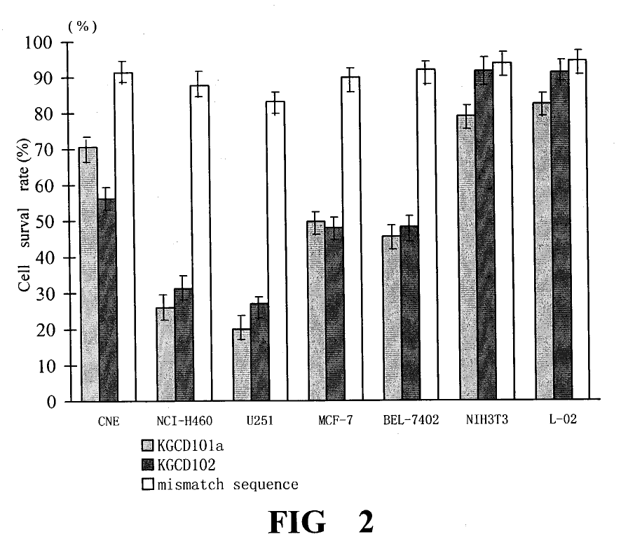

FIG. 3 is a table illustrating IC₅₀ of NCI-H460 tumor cells by nucleic acid decoys K101a and K102. Tumor cell line NCI-H460 is treated with different concentrations of K101a and K102. As shown in FIG. 3, K101a and K102 can inhibit the growth of NCI-H460 at relatively low concentrations with IC₅₀ around 30-50 nM. The negative control group shows no dose dependent effect on cell growth.

FIG. 4 is a table illustrating tumor volume changes of nude mice inoculated with human lung cancer cell line NCI-H460 after treatment by AK102. Groups of decoy drugs with different concentrations and formulations exhibit tumor growth inhibition to difference degrees 7 days after drug administration. The liposome group with 2.5 mg/kg concentration shows the highest growth inhibition of 77% (V/V) after 20 days of drug administration.

FIG. 5 is a diagram illustrating gel shift assay with K102 in polyacrylamide gel electrophoresis. Panel 1, 3: Complex formed between ³²P-K102 and cell nucleus extracts, Panel 2, 4: Complex formed between ³²P-K102 and cell nucleus extracts in the presence of unlabeled K102 (as specific competitor), Panel 5: ³²P-K102 only, no cell nucleus extracts. Results indicate K102 formed specific complex with nuclear proteins.

### DETAILED DESCRIPTION OF THE PREFERRED EMBODIMENT

In one preferred embodiment, therapeutic K101A series are designed to regulate the overexpression of the proto-oncogene C-FOS. Since the transcription factor CREB binds to the proto-oncogene C-FOS' enhancer CRE sequence STGACGTMR (seq. 1, SEQ ID NO: 1), seq. 1 can be used as the core sequence of nucleic acid decoys to competitively bind to CREB and regulate the expression levels of the proto-oncogene C-FOS. For example, K101a (seq. 6, SEQ ID NO: 6) is a single-stranded 23-mer oligonucleotide that forms a hairpin structure. K101a-1 is composed of a cruciform through the binding of seq. 7 (SEQ ID NO: 7) and seq. 8 (SEQ ID NO: 8) that contain said core sequence. K101a-2 is a double stranded linear structure formed by seq. 9 (SEQ ID NO: 9) that contains the core sequence. In summary, the therapeutic K101A series are single or double stranded DNA molecules 15-45 nucleotides long that contain the core sequence STGACGTMR.

In another preferred embodiment, the therapeutics K101b and K101c are designed to regulate the expression of multiple proto-oncogenes. The AP-1 transcription factor is a heterodimer of JUN/FOS that binds to the sequence STGASTMA (seq. 2, SEQ ID NO: 2) that is a cis-element to multiple genes and activates the expression of these genes. Said sequence is used as the core sequence in the nucleic acid decoy which can down-regulate the expression levels of the genes that contain the AP-1 enhancer. For example, K101b is a double stranded DNA molecule that is formed by_seq. 10 (SEQ ID NO: 10) that are 20-mer oligonucleotides containing said core sequence. K101b-1 (seq. 11, SEQ ID NO: 11) is a hairpin structure formed by a single stranded 21-mer oligonucleotide containing said core sequence. K101b-2 (seq. 12, SEQ ID NO: 12) is a 42-mer oligonucleotide that contains said core sequence. A ligase is used to form a closed loop by link K101b-2's 5′ end with its 3′ end. The resulting dumbbell structure enhances the drug's stability. K101c1 (seq. 13, SEQ ID NO: 13) and K101c2 (seq. 14, SEQ ID NO: 14) is comprised of 22-mer and 21-mer single-stranded oligonucleotides, respectively; K101c3 (seq. 15, SEQ ID NO: 15) and K101c4 (seq. 16, SEQ ID NO: 16) is comprised of 23-mer and 21-mer single-stranded oligonucleotides, respectively. All four therapeutics form the hairpin structure or become double stranded through self annealing and all contain said core sequence. In summary, the therapeutics K101b and K101c series are single or double stranded DNA molecules 15-45 nucleotides long that contain the sequence STGASTMA.

In another preferred embodiment, the therapeutic K102 series are design to use TTACCTCA (seq. 3, SEQ ID NO: 3) as the core sequence of the nucleic acid decoy to regulate the expression of the proto-oncogenes such as C-JUN. Said sequence is the conserved enhancer sequence that C-JUN binds to enhance its own expression. At the same time, the JUN/ATF protein heterodimer as a transcription factor also binds to said sequence to regulate the expression of the genes that have said sequence as an enhancer. For instance, K102 (seq. 17, SEQ ID NO: 17) is a single stranded 23-mer oligonucleotide hairpin structure that contain said core sequence. K102-1 (seq. 18, SEQ ID NO: 18) is a 21-mer oligonucleotide hairpin structure, K102-2 (seq. 19, SEQ ID NO: 19) is a 22-mer oligonucleotide, K102-3 (seq. 20, SEQ ID NO: 20) is a single stranded 20-mer oligonucleotide and K102-4 (seq. 21, SEQ ID NO: 21) is a linear double-stranded 20-mer nucleotide. In summary, the therapeutic K102 series are single or double stranded DNA molecules 15-45 nucleotides long that contain the sequence TTACCTCA.

In another preferred embodiment, the therapeutic K103a is designed to regulate the expression of proto-oncogene C-MYC. Since C-MYC binds to its own enhancer TCTCTTA (seq. 4, SEQ ID NO: 4), nucleic acid decoy drugs are designed to incorporate said core sequence to down regulate the expression levels of C-MYC. For example, K103a is a double stranded DNA comprised of seq. 22 (SEQ ID NO: 22), both of which contain said core sequence. K103a-1 (seq. 23, SEQ ID NO: 23) is a 25-mer oligonucleotide hairpin structure that contains said core sequence. 103a-2 is a 43-mer oligonucleotide stem-loop structure comprised of seq. 24 (SEQ ID NO: 24) and seq. 25 (SEQ ID NO: 25), both of which contain said core sequence. K103a-3 is a 36-mer oligonucleotide hammer structure comprised of seq. 26 (SEQ ID NO: 26) and seq. 27 (SEQ ID NO: 27) both of which contain said core sequence. In summary, the therapeutic K103a series are single or double stranded DNA molecules 15-45 nucleotides long that contain the sequence TCTCTTA.

In another preferred embodiment, the therapeutic K103b is designed to regulate the expression levels of genes associated with the cell cycle regulation. The protein heterodimer MYC/MAX, through binding to the cis-element RACCACGTGGTY (seq. 5, SEQ ID NO: 5), causes cell proliferation and the loss of cell cycle control. Nucleic acid decoy drugs are designed by incorporating said sequence as the core sequence. After they are synthesized in vitro and introduced into the cells, said drugs compete with the said cis-element for the binding of MYC/MAX heterodimer, leading to the repression of the down stream gene expression and suppression of the malignant growth phenotype. For example, K103b (seq. 28, SEQ ID NO: 28) is a 24-mer oligonucleotide containing said core sequence. K103b-1 (seq. 29) and K103-1 (seq. 30) are 24-mer single-stranded oligonucleodes that can self-anneal to form double-stranded dimer and that also contain said core sequence. In summary, the therapeutic K103b series are single or double stranded DNA molecules 15-45 nucleotides long that contain the sequence RACCACGTGGTY.

The nucleic acid decoy drugs are synthesized by automatic DNA synthesizer. After phosphorothioate, de-protection, purification and lyophilization, they are dissolved in water for qualitative and quantitative analysis. NCI-H460 lung cancer cell lines are used for in vitro functional screening and efficacy testing. The said cells are added to 96-well Petri dishes and incubated for 24 hours with the complete culture medium RPMI 1640. The nucleic acid decoy drug mixed with liposome is added after the incubation and the cells are further incubated at 37° C., 5% CO₂ for another 72 hours. The cell survival rate is determined by SRB colorimetry. The screening results indicate that all six series of nucleic acid decoy drugs can inhibit the growth of NCI-H460 to different degrees with K101a and K102 showing the most severe inhibition. All these nucleic acid decoys can be developed into anti-cancer therapeutic drugs.

The pharmaceutical composition claimed above acts on AP-1, CREB, MYC/MAX and JUN/ATF as novel targets of tumor therapies. Said protein molecules are valuable druggable targets due to their involvement in the regulation of the expression levels of multiple genes and are highly correlated with tumorigenesis.

Among the many potential methods to develop therapeutics against said drug targets, the nucleic acid transcription regulation decoys employed in this invention possess the advantages of a novel mechanism of action, clearly defined and specific functional targets with specific drug sequences. Said decoy drugs have been shown to effectively inhibit tumor cell growth in vitro experiments. These nucleic acid decoys are much smaller molecules that can be easily formulated into deliverable drugs when compared to gene therapy tumor treatments. Its advantage over anti-sense drugs lies in its double-stranded structure, which is more stable in vivo than single stranded anti-sense drugs. In addition, decoy drugs require less quantity for efficacy and act on totally new targets. As a result, said decoy drugs qualify as novel therapeutic entities.

The core sequences of said nucleic acid decoy drugs are 6-12 nucleotides long. These short sequences are easily degraded. The double stranded structures they form are not stable and easily denatured in room temperatures. In addition, the lack of two-dimensional structure makes them less likely to bind to proteins. To overcome these shortcomings, decoy drugs are improved by adding more nucleotides to elongate the chains for the purpose of increased stability and affinity to transcription factors. Considering the hairpin, cruciform, stem and loop and dumbbell structures are the favorable secondary structures, said nucleic acid decoy drugs are designed to contain the said core sequences with total length of 15-45 nucleotides. The resulting sequences can stay as single stranded, self anneal or hybridize to form various secondary structures. To avoid degradation by ribonucleases in vivo and enhance stability, the decoy drug sequences are chemically modified, and phosphorothioate is the most common protection method. In addition, in order to increase decoy molecules' membrane penetration and bioavailability, lipophilic groups, such as cholesterol are attached to either or both ends of the decoy molecules. The liposome method can also be used for the same purpose. Furthermore, targeting molecules, such as antibodies can also be attached to increase efficacy by concentrating the decoys preferentially in the tumors.

In conclusion, pharmaceutical composition of nucleic acid decoy molecules with AP-1, CREB, MYC/MAX AND JUN/ATF as the drug targets are described. Said decoys are shown to inhibit tumor cell growth and possess great promises as anti-cancer drug candidates. The said pharmaceutical composition can be combined with carriers consisted of saline, buffer, liposome and polymers to be made into formulations consisted of injectables, oral capsules and tablets, bolus, suppository and skin applicants. Said pharmaceutical composition can also be used in combination with chemotherapeutics and radiation therapies to enhance their efficacies.

### EXAMPLES

The following examples are intended to illustrate, but not limit, the scope of the invention.

### Example 1

### Growth Inhibition of Lung Cancer Cell Line NCI-H460 by 6 KGCD Series Nucleic Acid Decoys

Six phosphorothioate nucleic acid decoys as shown below are synthesized: K101a (seq. 6), K101b (seq. 10), K101c1 (seq. 13), K102 (seq. 17), K103 (seq. 22) and K103b (seq. 28).

| | |
|---|---|
| Negative control sequence: | 5′-TGTGGTCATGTGGTCATGTGT |
| | CA-3′ |
| Positive control sequence: | 5′-TGACGTCATGACGTCATGACG |
| | TCA-3′ |

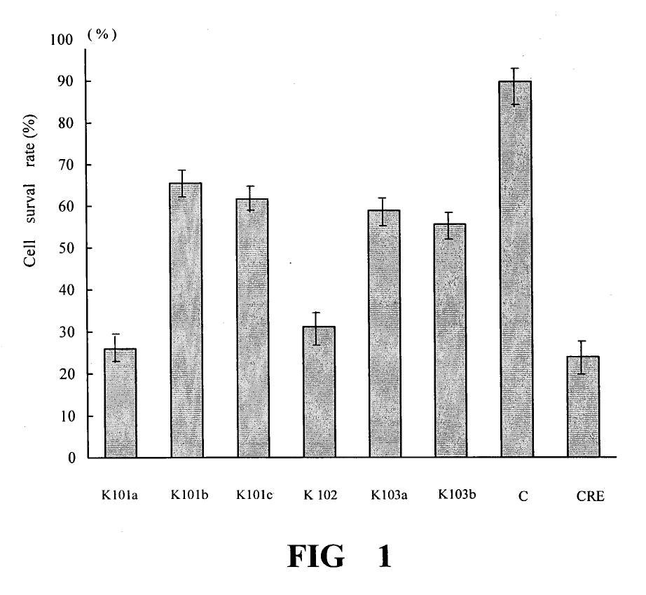

The lung cancer cell line NCI-H460 is divided into positive control, negative control, blank control and the six KGCD series nucleic acid decoy experimental groups. The cells are transferred into a 96 well Petri dish containing RPMI 1640 with 10% fetal bovine serum at 2-3×10⁴/well. After incubating for 24 hours, the culture medium is changed to RPMI 1640 alone. 0.6 pt/well liposome and a final concentration of 200 nM of the various nucleic acid decoys are added (for blank control, only 0.6 μL/well liposome is added). After 5 hours, the medium is changed to RPMI 1640 with 10% fetal bovine serum. The cells are further incubated 37° C. and 5% CO₂. After 72 hours, the cells are stained with SRB and the cell survival rates are determined by the OD values measured at 510 nm with a calorimeter. A cell growth inhibition graph is plotted with the blank control as 100%. Results are shown in FIG. 1: NCI-H460 cell growth is inhibited to various degrees by the decoys, with K101a and K102 exhibiting the most severe growth inhibition.

### Example 2

### Growth Inhibition of Various Tumor Cell Lines by Nucleic Acid Decoy K101a and K102.

The nucleic acid decoys are incubated with the following tumor cell lines: NCI-H460 (lung cancer), CNE (head and neck cancer), U251 (neuroglioblastoma cell), MCF-7 (breast cancer), BEL-7402 (liver cancer) and normal cell lines L-02 (liver cell) and NIH3T3. The column with the faintest color represents the negative control (scrambled sequence), showing little influence on cell growth. K101a and K102 inhibit the growth of all the tumor cell lines to different degrees with the growth inhibition to U251 and NCI-H460 the most severe. The decoys have little effective on the growth of normal cells.

### Example 3

### IC₅₀ of NCI-H460 tumor cells by nucleic acid decoy K101a and K102

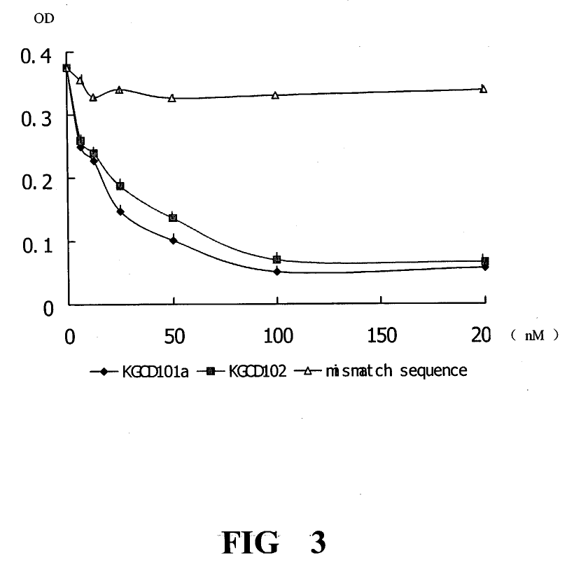

Tumor cell line NCI-H460 is treated with different concentrations of K101a and K102. After certain periods of incubation, OD values are measured to determine the cell survival rates. As shown in FIG. 3, K101a and K102 can inhibit the growth of NCI-H460 at relatively low concentrations with IC₅₀ around 30-50 nM, or 30-50 μg/ml, far higher than the anti-cancer drug screening standard (IC50<10 μg/ml).

### Example 4

### Anti-Cancer Efficacy Study of the Therapeutic K102 in vivo

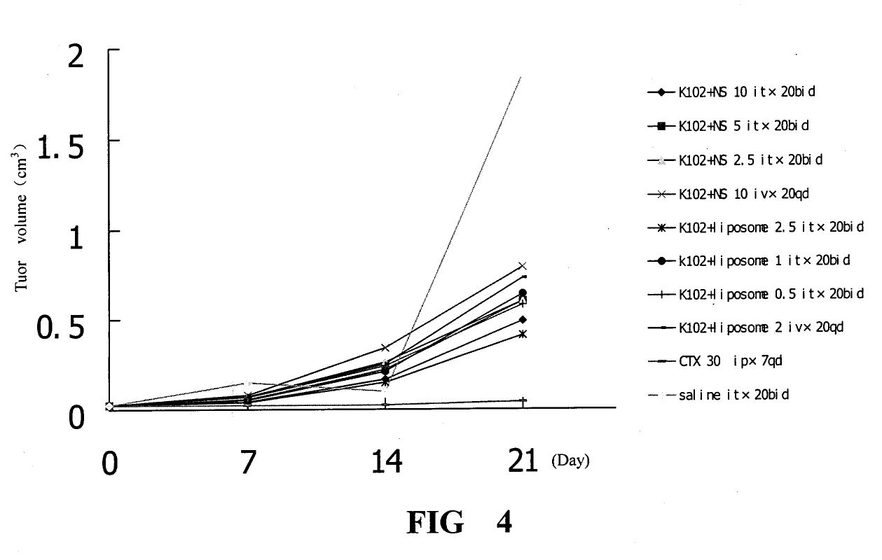

The therapeutic K102 is formulated with saline and liposome. 24 hours after T-cell deficient nude mice were inoculated with the tumor cell line NCI-H460 subcutaneously, K102 is administered daily into the tumor or i.v. at these doses: 10 mg/kg, 5 mg/kg and 2 mg/kg for the saline formulation and 2.5 mg/kg, 1 mg/kg and 0.5 mg/kg for the liposome formulation. Saline and pure liposome are used as controls. The tumor volume is measured dynamically. After 20 days of continuous drug administration, the animals are euthanized and the tumors are dissected and weighed. In the saline formulation group with it X 20 bid, tumor growth is inhibited by 73.5%, 67.57% and 67.57% (V/V), respectively while the i.v. X 20 qd group achieved tumor inhibition of 57.3% (V/V). For the liposome formulation group with it X 20 bid, tumor growth is inhibited by 77.84%, 65.41% and 68.65% (V/V) while the i.v. X 20 qd group achieved 60.55% (V/V) tumor inhibition. FIG. 4 shows the tumor volume dynamic changes of nude mice inoculated with human lung cancer cell line NCI-H460 after treatment with K102. It demonstrated that K102 is an effective inhibitor of tumor growth, independent of formulation.

### Example 5

Gel Shift Assay

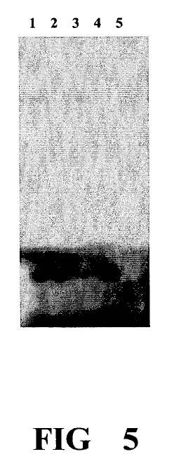

Nuclear extracts are prepared by the method of Digna et al. Oligonucleotides are ³²P-labeled with [γ³²P]ATP, using polynucleotide kinase. Binding reaction mixture containing 0.5 ng probe, 5 μg nuclear extract, 2 μg of poly (dI-dC) (to inhibit non-specific binding of K102 with nuclear proteins) and binding buffer [10 mM Tris (pH 7.5), 50 mM NaCl, 1 mM DTTI, 1 mM EDTA, 5% glycerol] is incubated for 20 minutes at the room temperature. The DNA-protein complexes are resolved by electrophoresis through 4% poly-acrylamide gels containing 50 mM Tris, 0.38 M glycine and 2 mM EDTA. The gels are subsequently dried and autoradiographed with intensifying screens at −70° C. Results shown in FIG. 5 indicate that K102 forms specific complex with nuclear proteins, providing the support for the mechanism of action for K102.

While a preferred embodiment of the present invention has been described in these Examples, it should be understood that various changes, adaptations and modifications may be made therein without departing from the spirit of the invention and the scope of the appended claims.

Having described the preferred embodiments of the present invention, it will appear to those ordinarily skilled in the art that various modifications may be made to the disclosed embodiments, and that such modifications are intended to be within the scope of the present invention. Although the invention has been described with reference to the above examples, it will be understood that modifications and variations are encompassed within the spirit and scope of the invention. Accordingly, the invention is limited only by the following claims.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure form such principles. Therefore, this invention includes all modifications encompassed within the spirit and scope of the following claims.

What is claimed is:

1. An isolated composition comprising a 15-45 oligonucleotide sequence having a double-stranded structure having affinity for binding a transcription factor in a host cell.

2. The composition, as recited in claim 1, wherein said polynucleotide sequence is selected from a group consisting of SEQ ID NO: 1 to 30.

3. The composition, as recited in claim 1, wherein said transcription factor is selected from a group consisting of CREB, AP1(JUN/FOS), JUN/ATF, C-MYC and MYC/MAX.

4. The composition, as recited in claim 1, wherein said host cell is a mammalian cell.

5. The composition, as recited in claim 4, wherein said mammalian cell is a human cell.

6. The composition, as recited in claim 5, wherein said human cell is a human cancer cell.

7. The composition, as recited in claim 1, wherein said polynucleotide is double-stranded.

8. The composition, as recited in claim 1, wherein said polynucleotide has a single-stranded structure which is able to form said double-stranded structure through an intra-chain interaction.

9. The composition, as recited in claim 1, wherein said polynucleotide has a single-stranded structure which is able to form said double-stranded structure through an inter-chain interaction.

10. The composition, as recited in claim 1, wherein said polynucleotide has a single-stranded structure which is able to, through an intra-chain interaction, form a secondary structure selectively comprising hairpin, pseudo-knot, stem and loop, dumb-bell and cruciform.

11. The composition, as recited in claim 1, wherein said polynucleotide has a single-stranded structure which is able to, through an inter-chain interaction, form a secondary structures selectively comprising hairpin, pseudo-knot, stem and loop, dumb-bell and cruciform.

12. The composition, as recited in claim 1, wherein said polynucleotide comprises a nucleotide analog.

13. The composition, as recited in claim 12, wherein said nucleotide analog is selected from a group consisting of deoxyuracil, labeled nucleotide, ribonucleotide, 7-deaza-dNTP, methylthio-linked nucleotide, phosphothio-linked nucleotide, morpholino nucleotide, hexose-containing nucleotide, peptide nucleic acid (PNA) and derivatives thereof.

14. The composition, as recited in claim 12, wherein said nucleotide analog is labeled and attached with a ligand molecule selected from a group consisting of lipophilic substrate, target-specific antibodies or other molecules with targeting functions and a combination thereof.

15. The composition, as recited in claim 1, wherein said polynucleotide is formulated into pharmaceutical formulation selected from a group consisting of injectable, oral, transdermal, bolus, aerosol, suppository, transfectional and transgenic formulations and a combination thereof.

16. An isolated composition comprising a 15-45 oligonucleotide comprising a double-stranded sequence 5′-STGACGTMR-3′ which is capable of binding to transcription factor CREB.

17. An isolated composition comprising a 15-45 oligonucleotide comprising a double-stranded sequence 5′-STGASTMA-3′ which is capable of binding to transcription factor AP-1 (JUN/FOS).

18. An isolated composition comprising a 15-45 oligonucleotide comprising a double-stranded sequence 5′-TTACCTCA-3′ which is capable of binding to transcription factor JUN/ATF.

19. An isolated composition comprising a 15-45 oligonucleotide comprising a double-stranded sequence 5′-TCTCTTA-3′ which is capable of binding to transcription factor C-MYC.

20. An isolated composition comprising a 15-45 oligonucleotide comprising a double-stranded sequence 5′-RACCACGTGGTY-3′ which is capable of binding to transcription factor MYC/MAX.

21. An isolated composition comprising an element selected from a group consisting of (a) a polynucleotide selected from a group consisting of a 15-45 oligonucleotide comprising a double-stranded sequence 5′-STGACGTMR-3′ which is capable of binding to transcription factor CREB, a 15-45 oligonucleotide comprising a double-stranded sequence 5′-STGASTMA-3′ which is capable of binding to transcription factor AP-1 (JUN/FOS), a 15-45 oligonucleotide comprising a double-stranded sequence 5′-TTACCTCA-3′ which is capable of binding to transcription factor JUN/ATF, a 15-45 oligonucleotide comprising a double-stranded sequence 5′-TCTCTTA-3′ which is capable of binding to transcription factor C-MYC, and a 15-45 oligonucleotide comprising a double-stranded sequence 5′-RACCACGTGGTY-3′ which is capable of binding to transcription factor MYC/MAX; (b) a polynucleotide of at least 70% homology to the polynucleotide of (a); and (c) a combination thereof.

22. The composition, as recited in claim 21, wherein said polynucleotide is double-stranded.

23. The composition, as recited in claim 21, wherein said polynucleotide has a single-stranded structure which is able to form said double-stranded structure through an intra-chain interaction.

24. The composition, as recited in claim 21, wherein said polynucleotide has a single-stranded structure which is able to form said double-stranded structure through an inter-chain interaction.

25. The composition, as recited in claim 21, wherein said polynucleotide has a single-stranded structure which is able to, through an intra-chain interaction, form a secondary structure selectively comprising hairpin, pseudo-knot, stem and loop, dumb-bell and cruciform.

26. The composition, as recited in claim 21, wherein said polynucleotide has a single-stranded structure which is able to, through an inter-chain interaction, form a secondary structure selectively comprising hairpin, pseudo-knot, stem and loop, dumb-bell and cruciform.

27. The composition, as recited in claim 21, wherein said polynucleotide comprising a nucleotide analog.

28. The composition, as recited in claim 27, wherein said nucleotide analog is selected from a group consisting of deoxyuracil, labeled nucleotide, ribonucleotide, 7-deaza-dNTP, methylthio-linked nucleotide, phosphoramidite, phosphothio-linked nucleotide, morpholino nucleotide, hexose-containing nucleotide, peptide nucleic acid (PNA) and derivatives thereof.

29. The composition, as recited in claim 28, wherein said nucleotide analog is labeled and attached with a ligand molecule selected from a group consisting of lipophilic substrate, target-specific antibodies or other molecules with targeting functions and a combination thereof.

30. The composition, as recited in claim 21, wherein said polynucleotide is formulated into pharmaceutical formulation selected from a groups consisting of injectable, oral, transdermal, bolus, aerosol, suppository, transfectional and transgenic formulations and a combination thereof.

31. A method for regulating gene transcription and cell growth in a target cell comprising the steps of:
a) providing one or more transcription regulatory element decoys each comprising a polynucleotide, wherein said polynucleotide is selected from a group consisting of SEQ ID NO: 1 to 30; and
b) exposing said target cell to said polynucleotide under a condition that said polynucleotide alters gene transcription and cell growth.

32. The method, as recited in claim 31, wherein the said target cell is a human cancer cell.

33. The method, as recited in claim 31, wherein said target cell is exposed to said polynucleotide by injection.

34. The method, as recited in claim 31, wherein said target cell is exposed to said polynucleotide by direct exposure.

35. The method, as recited in claim 31, wherein said target cell is exposed to said polynucleotide by oral intake.

36. The method, as recited in claim 31, wherein said target cell is exposed to said polynucleotide by transfection.

37. The method, as recited in claim 31, wherein said target cell is exposed to said polynucleotide by transgenic expression.
